(19) United States
(12) Patent Application Publication
(10) Pub. No.: US 2009/0266907 A1
(43) Pub. Date: Oct. 29, 2009

(54) Volatile Liquid Emission Apparatus

(76) Inventors: Colin Litten-Brown, Bracknell (GB); Guy Edward Naish, Bicester (GB)

Correspondence Address: NORRIS, MCLAUGHLIN & MARCUS, 875 THIRD AVE, 18TH FLOOR, NEW YORK, NY, 10022, US

(21) Appl. No.: 12/441,830

(22) PCT Filed: Sep. 20, 2007
(86) PCT No.: PCT/CH07/00462

§ 371 Date: Mar. 18, 2009

(30) Foreign Application Priority Data
Sep. 26, 2006 (GB) 0618856.9

(51) Int. Cl. A61L 9/04

(52) U.S. Cl. 239/1; 239/44

(57) ABSTRACT

An apparatus adapted to disseminate a volatile liquid, for example, a fragrance, into an atmosphere, the apparatus comprising a reservoir containing the liquid and a porous liquid transfer member through which the liquid is conveyed from the reservoir to the atmosphere and from which part extending into the atmosphere it is disseminated, the reservoir consisting of a collapsible bladder that reduces in volume responsive to atmospheric pressure and the loss of liquid, the porous transfer member being encased in an impermeable sleeve that is open to the liquid at that end of the member remote from the that part in the atmosphere, the transfer member additionally comprising at or adjacent to the open end means that interact with the collapsing bladder to maintain liquid contact with the transfer member.

The apparatus is a simple and effective device that substantially solves the problems of leakage and complete utilisation of the volatile liquid,

This invention relates to apparatus for disseminating volatile liquids into an atmosphere.

The presence in an atmosphere of a volatile liquid, such as a fragrance, an insecticide, a fungicide or a disinfectant, is often desired. A common way of achieving this is by means of a simple device comprising a reservoir containing the liquid and a porous liquid transfer member that serves both to remove the liquid from the reservoir and disseminate it into the atmosphere. The transfer member is typically a wick of any suitable material, such as plastics, ceramics and natural fibres. Such apparatus have found widespread acceptance.

In order for such an apparatus to function, it must have a means of pressure equalisation between reservoir and atmosphere, so that liquid dissemination can be continuous; without such means, the pressure in the reservoir would drop with loss of liquid and dissemination would stop long before the liquid is exhausted. Another aspect of pressure equalization is the reverse, excessive pressure inside the reservoir. This has the potential to occur when an apparatus is exposed to heat, for example, direct sunlight. Without a means of pressure equalization, the resultant increased pressure in the reservoir can force liquid into the transfer member at a rate that is higher than desirable and faster than it can be evaporated, meaning that it pools at the atmosphere end of the transfer member, a potential source of leakage.

One typical way is to provide a small passage joining the atmosphere with the interior of the reservoir. In another method, the transfer member itself can be made sufficiently porous, so that the pressure can be equalised. Such means are perfectly adequate for those apparatus that will remain in a single orientation for their entire service lives. However, should this not be the case, there can be problems. For example, one type of air freshener that can disseminating two fragrances has the form of two conventional, independent, wick-type air fresheners mounted in 180° opposition to each other and joined at what would normally be considered their upper ends. Such two-fragrance air fresheners often resemble an hourglass, and like an hourglass they can be inverted and stood stably on a base at either end, the uppermost end becoming the fragrance disseminator via a dissemination surface connected to or forming part of the wick of the uppermost reservoir and positioned at or near the base. Dissemination from the wick of the uppermost reservoir only is ensured by partially encasing the wicks in both reservoirs in sleeves, such that the wick in the lower portion is shielded from liquid. A fragrance may therefore be changed by simply inverting the device. It will readily be understood that the normal means of providing pressure equalization will not work in this case, as a conventional vent will allow the liquid to run out.

One way of ensuring pressure equalisation without leakage is to seal the liquid in an impermeable, collapsible bladder. As liquid is disseminated, the bladder collapses, thus equalising the pressure. This, however, can cause the bladder to collapse around the wick, blocking it and reducing liquid dissemination, or even blocking it altogether. This occurs most frequently with the abovementioned sleeved wicks.

It has now been found that these problems may be substantially of completely overcome by a particular construction. There is therefore provided an apparatus adapted to disseminate a volatile liquid into an atmosphere, the apparatus comprising a reservoir containing the liquid and a porous liquid transfer member through which the liquid is conveyed from the reservoir to the atmosphere and from which part extending into the atmosphere it is disseminated, the reservoir consisting of a collapsible bladder that reduces in volume responsive to atmospheric pressure and the loss of liquid, the porous transfer member being encased in an impermeable sleeve that is open to the liquid at that end of the member remote from the that part in the atmosphere, the transfer member additionally comprising at or adjacent to the open end means that interact with the collapsing bladder to maintain liquid contact with the transfer member.

The invention further provides a method of disseminating into an atmosphere substantially the entirety of a volatile liquid contents in a reservoir having the form of a collapsible bladder sealed from the atmosphere and disseminating the liquid by means of a porous liquid transfer member extending from the reservoir into the atmosphere, comprising providing that portion of the transfer member with a liquid-impermeable sleeve open to the liquid at or near that end remote from the atmosphere, the transfer member additionally comprising at or adjacent to the open end means that interact with the collapsing bladder to maintain liquid contact with the transfer member.

The reservoir is a flexible bladder that is capable of changing volume as liquid is disseminated. For example, it may be somewhat like a bladder or balloon of a material that is deformable. Depending on the nature of the apparatus, this deformation may be elastic or non-elastic. Thus the material may be elastomeric (able to return to its original shape) when a force causing distortion is removed, or non-elastomeric (a material that can be stretched, but which remains permanently in the stretched state). In one embodiment, the bladder may be filled with liquid, and it progressively collapses as liquid is withdrawn, but which can also expand if the internal pressure increases above atmospheric pressure. This bladder may be housed in a suitable support structure that provides a base for standing, such as a rigid vessel or a cage.

In the case of non-elastomeric bladders, there can be provided a folded or “concertina” structure, in which the reservoir can progressively expand with increasing external temperature or collapse as liquid is removed, the folds or concertina elements opening out or narrowing (possibly with concomitant elastomeric or non-elastomeric expansion or contraction of the material).

The nature of the liquid and the end-use will be decisive as to what kind of reservoir in desirable, and the skilled person can generally decide on an appropriate embodiment for any given case by simple experimentation. However, in the case of fragrances for air fresheners and the like, there are some important considerations to be borne in mind. Such materials are non-aqueous and very volatile, so the reservoir must be resistant to the contents, in the sense that it is not physically or chemically degraded by the contents. It must also contain the contents without appreciable loss. Many conventional materials otherwise suitable for reservoirs do not meet these conditions.

In such cases, the preferred materials are plastics laminates, that is, layers of plastics materials that have been laminated together to form a chemically-resistant and impermeable film. These are commonly found in the food packaging industry and typical examples include PA/PE, PA/EVOH/PE, PET/PE/EVOH/PE, PA/EVOH/EVA/PA and PET/Al/PE where PA=polyamide, PE=polyethylene, EVOH=ethylene-vinyl alcohol copolymer, PET=polyethylene terephthalate, EVA=ethylene-vinyl acetate copolymer and Al=aluminium foil.

Such materials are flexible, but not elastomeric, so the reservoirs must be constructed such that they have the ability to reduce in volume. This may be done, for example, by providing the reservoirs with pleats, flutes or folds, such that a full reservoir has an outer surface substantially or completely without folds, but as liquid is disseminated, the surface folds and the reservoir loses volume.

In a further embodiment, the reservoir material is sufficiently transparent or translucent so that the quantity of liquid therein may be observed and therefore the need for replacement may be assessed. The selection and utilisation of a suitable material is well within the skill of the art.

A particular material for the purposes of this invention is a PA/EVOH/EVA/PA laminate of from 50-100 micrometres thickness. This particular material is transparent. Although a range of thicknesses can be used, thickness for particular use with fragrances are generally from about 50 to about 100 micrometres, in particular about 60 micrometres. This gives adequate foldability and physical strength. A typical suitable commercially-available material is Amilen™ OX-80 ex Nabenhauer Verpackungen GmbH, Germany.

The liquid transfer member may be any suitable member. For example, it may be a porous wick of any suitable material, for example, compressed natural or artificial fibre, plastics, ceramics or metal. It may be moulded or stamped out of a sheet of material.

The liquid transfer member is supplied with a sleeve that is impermeable to the liquid and that allows the liquid to contact the transfer member only at or near that end of the transfer member remote from the end at which dissemination takes place. While the sleeve variant may be used with any variant of the apparatus, it is especially useful with any variant in which the device is inverted and the liquid cannot then be disseminated, for example, the hourglass-type air freshener hereinabove mentioned. The sleeve may be made of any suitable material and fitted by any convenient means. For example, it may be a pre-manufactured sleeve that is fitted to the transfer member, or it may be an impermeable coating applied to all but the end of the member.

The transfer member additionally comprises, at or adjacent to the open end, means that interact with the collapsing bladder to maintain liquid contact with the transfer member.

This may be any suitable means that stops the bladder from cutting off the open end from the supply of liquid as it collapses. It may be an additional component added to the transfer member. Alternatively, it may be part of the sleeve. If the sleeve is made of plastics material, the necessary means may be formed in the sleeve before it is fitted.

There are many possibilities and the skilled person will readily be able to conceive of them. Some specific, non-limiting embodiments include:
a flange or a series of projections extending radially from the end of the sleeve and capable of holding the collapsing bladder away from the open end;
a series of ports provided around the circumference, optionally each port being in contact with a longitudinal groove formed in the surface of the sleeve, the ports and/or grooves being suitably dimensioned and located that blockage is not possible;
a cage formed in the end of the sleeve and extending clear of the end of the transfer means;
a series of longitudinal ridges formed on the surface of the sleeve in contact with the liquid.

Although the invention may be used in connection with any such device, it is particularly useful in devices in which conventional vented construction is not possible, for example the hourglass-type apparatus hereinabove described, in which the apparatus may be inverted. The invention therefore additionally provides an apparatus adapted to disseminate into an atmosphere one of two volatile liquids, the apparatus having, in use configuration, an upper and a lower reservoir, each containing one liquid and each equipped with a porous liquid transfer member that extends from the reservoir to the atmosphere, the transfer member being provided with an impermeable sleeve such that only that end of the transfer member remote from the end exposed to the atmosphere is open to the liquid, the apparatus being invertible, each reservoir consisting of a collapsible bladder that reduces in volume responsive to atmospheric pressure and the loss of liquid, and the transfer member additionally comprising, at or adjacent to the end open to the liquid, means that interact with the collapsing bladder to maintain liquid contact with the transfer member.

The structure of the apparatus is not critical, provided that the possibility of providing an upper and a lower reservoir is achievable. This may be achieved by any convenient means. Examples of such means include:
pivotally mounting the reservoirs on a base, such that the desired reservoir can be rotated into position;
providing a rigid receptacle, such as a container or a cage, within which a flexible reservoir may be mounted.

The skilled person will readily appreciate the various possibilities and how they can be carried out.

When a reservoir is the lower reservoir, no liquid contacts the transfer member, and there is no liquid transfer. When the apparatus is inverted and the lower reservoir becomes the upper reservoir, the liquid again contacts that part of the transfer member that is not covered by the sleeve and liquid can again move up the transfer member for dissemination into the atmosphere.

Any potential for leakage at the time of a new inversion can be easily handled by conventional means, for example, by providing the lower reservoir adjacent to the liquid dissemination surface with a lid or a tray. This will cope with any residual liquid, which will soon evaporate and no more will be forthcoming, until re-inversion.

Apparatus according to this invention are easily manufactured using known materials and techniques, and they work very effectively. They may be made as single-use apparatus, or the reservoirs may be supplied as refills ready for insertion into an apparatus. They may also be made refillable by, for example, removing the transfer means, inverting the reservoir and adding liquid through a tube that extends into the reservoir.

The invention will now be further described with reference to the drawings, which depict a preferred embodiment and which is not intended to be in any way limiting.

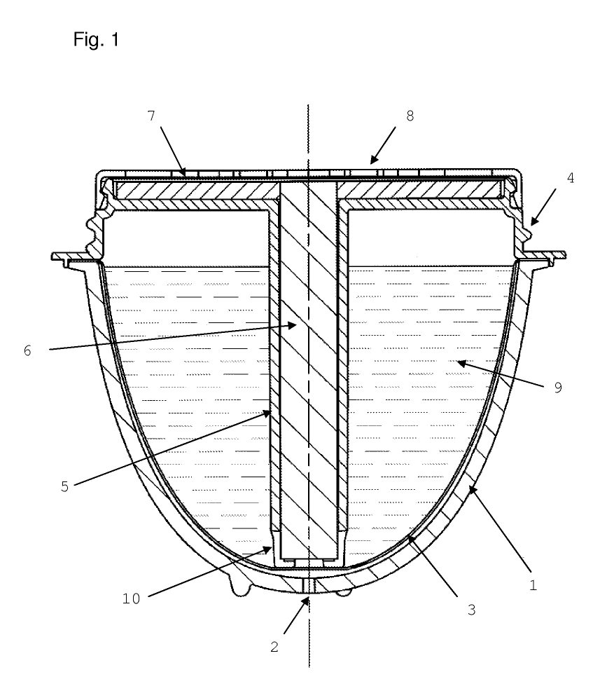

FIG. 1 is a vertical cross-section through an embodiment.

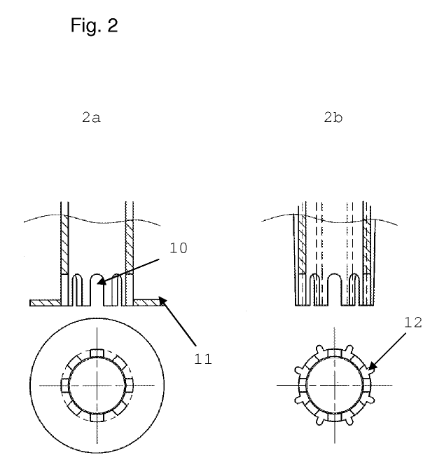

FIG. 2 represents a series of longitudinal and transverse cross-sections through two sleeves according to the invention.

In FIG. 1, a container of parabolic vertical cross-section 1 has a vent hole 2 at its base. Within the container is a reservoir that is a flexible bag 3, secured in place by a cap 4. The cap comprises a centrally-placed, vertical, downwardly-extending sleeve 5, within which is a tightly-fitting porous wick 6. At the upper end, this wick makes liquid transfer contact with a flat liquid emanator 7, which is exposed to the atmosphere and which is covered by a protective grid 8. The reservoir contains a volatile liquid 9, which is to be disseminated into the atmosphere.

In the lower end of the sleeve 5 are formed a plurality of arch-shaped slots 10, which allow liquid to reach the wick 6, but which prevent the bladder collapsing completely and blocking the access of the liquid to the wick.

In operation when upright, the volatile liquid 9 is transferred up the wick 6 through capillary action and the pressure is equalized by the reservoir 3 collapsing inwards towards the sleeve 5. The vent hole 2 in the protective rigid container 1 allows equalization of pressure outside the bag 3.

In FIG. 2, there can be seen three embodiments of means for ensuring that the collapsing bladder does not prevent liquid from reaching the wick. In FIG. 2a, there extends from the sleeve a radial flange 11, which holds the bladder away from the slots 10. In FIG. 2b, the sleeve additionally comprises a series of longitudinal ridges.

The skilled person will readily appreciate that many variations of this invention can be made by simply applying the ordinary skill of the art, and that the previously described variants are by way of example only and are not meant to be limiting on the scope of the invention in any way.

1. An apparatus adapted to disseminate a volatile liquid into an atmosphere, the apparatus comprising a reservoir containing the liquid and a porous liquid transfer member through which the liquid is conveyed from the reservoir to the atmosphere and from which part extending into the atmosphere it is disseminated, the reservoir consisting of a collapsible bladder that reduces in volume responsive to atmospheric pressure and the loss of liquid, the porous transfer member being encased in an impermeable sleeve that is open to the liquid at that end of the member remote from the that part in the atmosphere, the transfer member additionally comprising at or adjacent to the open end means that interact with the collapsing bladder to maintain liquid contact with the transfer member.

2. An apparatus according to claim 1, in which the bladder is made of elastomeric material.

3. An apparatus according to claim 1, in which the bladder is of non-elastomeric material and comprises a folded or concertina structure adapted to permit volume change.

4. An apparatus according to claim 1, in which the bladder is sufficiently transparent or translucent to allow liquid therein to be seen.

5. An apparatus according to claim 3, in which the material is a flexible plastic laminate.

6. An apparatus according to claim 5, in which the laminate is selected from the group consisting of the following materials:
polyamide/polyethylene,
polyamide/ethylene-vinyl alcohol copolymer/polyethylene,
polyethylene terephthalate/polyethylene/ethylene-vinyl alcohol copolymer/polyethylene,
polyamide/ethylene-vinyl alcohol copolymer/ethylene-vinyl acetate copolymer/polyamide and
polyethylene terephthalate/aluminium foil/polyethylene.

7. An apparatus according to claim 6, in which the material is a polyamide/ethylene-vinyl alcohol copolymer/ethylene-vinyl acetate copolymer/polyamide laminate of from 50-100 micrometres thickness.

8. An apparatus according to claim 1, in which the means that interact with the collapsing bladder to maintain liquid contact with the transfer member is selected from the group consisting of:
a flange or a series of projections extending radially from the end of the sleeve and capable of holding the collapsing bladder away from the open end;
a series of ports provided around the circumference, optionally each port being in contact with a longitudinal groove formed in the surface of the sleeve, the ports and/or grooves being suitably dimensioned and located that blockage is not possible;
a cage formed in the end of the sleeve and extending clear of the end of the transfer means;
a series of longitudinal ridges formed on the surface of the sleeve in contact with the liquid.

9. An apparatus adapted to disseminate into an atmosphere one of two volatile liquids, the apparatus having, in use configuration, an upper and a lower reservoir, each containing one liquid and each equipped with a porous liquid transfer member that extends from the reservoir to the atmosphere, the transfer member being provided with an impermeable sleeve such that only that end of the transfer member remote from the end exposed to the atmosphere is open to the liquid, the apparatus being invertible, each reservoir consisting of a collapsible bladder that reduces in volume responsive to atmospheric pressure and the loss of liquid, and the transfer member additionally comprising, at or adjacent to the end open to the liquid, means that interact with the collapsing bladder to maintain liquid contact with the transfer member.

10. A method of disseminating into an atmosphere substantially the entirety of a volatile liquid contents in a reservoir having the form of a collapsible bladder sealed from the atmosphere and disseminating the liquid by means of a porous liquid transfer member extending from the reservoir into the atmosphere, comprising providing that portion of the transfer member with a liquid-impermeable sleeve open to the liquid at or near that end remote from the atmosphere, the transfer member additionally comprising at or adjacent to the open end means that interact with the collapsing bladder to maintain liquid contact with the transfer member.
